(19) United States
(12) Patent Application Publication
(10) Pub. No.: US 2014/0296821 A1
(43) Pub. Date: Oct. 2, 2014

(54) Absorbent Article

(71) Applicant: KIMBERLY-CLARK WORLDWIDE, INC., Neenah, WI (US)

(72) Inventors: Susan Kay Bronk, Greenville, WI (US); Heidi Kay Wudi, Appleton, WI (US); Nancy Ellen Dawson, Neenah, WI (US)

(73) Assignee: Kimberly-Clark Worldwide, Inc., Neenah, WI (US)

(21) Appl. No.: 13/853,756

(22) Filed: Mar. 29, 2013

(51) Int. Cl. A61F 13/56; A61F 13/62

(52) U.S. Cl. CPC A61F 13/5655; A61F 13/625
USPC 604/391

(57) ABSTRACT

An absorbent article which can be applied to a wearer in a standing position. The absorbent article can be adjusted following the placement of the absorbent article on the wearer. The absorbent article can have a back fastening system and a front fastening system. The front fastening system can have a bilobate ear portion wherein each lobe can be independently adjusted.

### BACKGROUND

Disposable absorbent articles are generally utilized to absorb and retain body exudates, such as urine and fecal material. Two types of absorbent articles suitable for wear around the lower torso of the wearer include conventional diaper designs and closed absorbent articles such as diaper pants, training pants and youth pants.

Conventional diaper designs are generally worn by small babies, people with incontinence, or people who are sick or otherwise confined to a bed. A conventional diaper is fitted onto a wearer by first placing a portion of the diaper under the wearer (e.g., the back waist region of the diaper is placed under the buttocks and back waist of the wearer) and then pulling the remainder of the diaper through the wearer's legs. The back waist region of the diaper is then attached to the outside of the front waist region of the diaper on each side of the wearer. The conventional diaper, however, is difficult to use when the wearer does not remain still throughout the period of application of the diaper to the wearer. Additionally, adult wearers also find conventional diapers difficult to put on themselves without assistance.

Closed absorbent articles, such as diaper pants, training pants, youth and adult pants, generally allow the wearer to pull the absorbent article on themselves as they would pants and these absorbent articles also generally do not require any form of fastening mechanism such as are found on conventional diapers. The closed absorbent article, however, can be difficult for a wearer to pull on or for a user to put on a wearer without removing the wearer's clothing. Additionally, once the absorbent article is on the wearer, little adjustment can be made to the absorbent article to ensure a proper fit of the absorbent article to the wearer which is desirable as proper fit can reduce or eliminate leakage of body exudates from the absorbent article.

There is a need for an absorbent article which can be more easily applied to the wearer of the absorbent article. There is a need for an absorbent article which can be applied to a wearer who is in a standing position. There is a need for an absorbent article which can be adjusted to fit snugly about the torso and legs of the wearer.

### SUMMARY

In an embodiment, an absorbent article can comprise a front waist region, a back waist region, and a crotch region interconnecting the front waist region and the back waist region; a garment facing surface and a body facing surface; a back fastening system located in the back waist region which can comprise a pair of structural members having a garment facing surface and a body facing surface and a fastener located on the garment facing surface of each of the structural members and configured to releasably engage the body facing surface in the front waist region of the absorbent article; a front fastening system located in the front waist region which can comprise a pair of bilobate ear portions having a garment facing surface and a body facing surface and a fastener located on the body facing surface of each of the ear portions. In an embodiment, the pair of ear portions of the front fastening system can be constructed from a non-elastic material. In an embodiment, the pair of ear portions of the front fastening system can be constructed from an elastic material. In an embodiment, the fastener of each of the structural members can comprise a hook fastener. In an embodiment, the fastener of each of the ear portions can comprise a hook fastener. In an embodiment, each lobe of each ear portion can comprise a fastener. In an embodiment, each of the structural members of the back fastening system can further comprise a stretch component. In an embodiment, the fastener of each of the ear portions can be configured to releasably engage the garment facing surface of the absorbent article in the back waist region of the absorbent article. In an embodiment, the fastener on each of the ear portions can be configured to releasably engage the garment facing surface of each of the structural members of the back fastening system.

In an embodiment, an absorbent article can comprise a front waist region, a back waist region, and a crotch region interconnecting the front waist region and the back waist region; a garment facing surface and a body facing surface; an outer cover and a body facing liner; a back fastening system located in the back waist region which can comprise a pair of structural members having a garment facing surface and a body facing surface and a fastener located on the garment facing surface of each of the structural members and configured to releasably engage the body facing surface in the front waist region of the absorbent article; a front fastening system located in the front waist region which can comprise a pair of bilobate ear portions having a garment facing surface and a body facing surface and a fastener at least partially located in each lobe of each ear portion and configured to releasably engage the outer cover. In an embodiment, the pair of ear portions of the front fastening system can be constructed from a non-elastic material. In an embodiment, the pair of ear portions of the front fastening system can be constructed from an elastic material. In an embodiment, each of the structural members of the back fastening system can further comprise a stretch component. In an embodiment, the fastener of each of the structural members can comprise a hook fastener. In an embodiment, each of the fasteners of each of the lobes of each of the ear portions can comprise a hook fastener.

In an embodiment, an absorbent article can comprise a front waist region, a back waist region, and a crotch region interconnecting the front waist region and the back waist region; a garment facing surface and a body facing surface; an outer cover and a body facing liner; a back fastening system located in the back waist region which can comprise a pair of structural members having a garment facing surface and a body facing surface and a fastener located on the garment facing surface of each of the structural members and configured to releasably engage the body facing surface in the front waist region of the absorbent article; a front fastening system located in the front waist region which can comprise a pair of bilobate ear portions having a garment facing surface and a body facing surface and a fastener at least partially located in each lobe of each ear portion and configured to releasably engage with the garment facing surface of each structural member. In an embodiment, the pair of ear portions of the front fastening system can be constructed from a non-elastic material. In an embodiment, the pair of ear portions of the front fastening system can be constructed from an elastic material. In an embodiment, each of the structural members of the back fastening system can further comprise a stretch component. In an embodiment, the fastener of each of the structural members can comprise a hook fastener. In an embodiment, each of the fasteners of each of the lobes of each of the ear portions can comprise a hook fastener.

### BRIEF DESCRIPTION OF THE DRAWINGS

FIG. 1 is a plan view of a non-limiting illustration of an absorbent article, such as, for example, a diaper, in an unfastened, stretched and laid flat condition with the surface of the absorbent article which contacts the wearer facing the viewer and with portions cut away for clarity of illustration.

FIG. 2 is a side view of a non-limiting illustration of the absorbent article of FIG. 1 in a closed configuration.

### DETAILED DESCRIPTION

In an embodiment, the present disclosure is generally directed towards an absorbent article which can be applied to a wearer in a standing position. In an embodiment, the present disclosure is generally directed towards an absorbent article which can be applied to a wearer with or without the wearer's clothes on. In an embodiment, the present disclosure is generally directed towards an absorbent article which can be adjusted about the torso and/or legs of the wearer following the placement of the absorbent article on the wearer.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads, incontinence products, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” refers herein to the joining, adhering, connecting, attaching, or the like, of two elements. Two elements will be considered bonded together when they are joined, adhered, connected, attached, or the like, directly to one another or indirectly to one another, such as when each is directly bonded to intermediate elements. The bonding of one element to another can occur via continuous or intermittent bonds.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “elastically extensible” refers herein to a material or combination of materials which can extend in at least one direction when a force is applied and return to approximately their original dimensions after the force is removed.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “fluid entangling” and “fluid entangled” refers herein to a formation process for further increasing the degree of fiber entanglement within a given fibrous nonwoven web or between fibrous nonwoven webs and other materials so as to make the separation of the individual fibers and/or the layers more difficult as a result of the entanglement. Generally this is accomplished by supporting the fibrous nonwoven web on some type of forming or carrier surface which has at least some degree of permeability to the impinging pressurized fluid. A pressurized fluid stream (usually multiple streams) can then be directed against the surface of the nonwoven web which is opposite the supported surface of the web. The pressurized fluid contacts the fibers and forces portions of the fibers in the direction of the fluid flow thus displacing all or a portion of a plurality of the fibers towards the supported surface of the web. The result is a further entanglement of the fibers in what can be termed the Z-direction of the web (its thickness) relative to its more planar dimension, its X-Y plane. When two or more separate webs or other layers are placed adjacent one another on the forming/carrier surface and subjected to the pressurized fluid, the generally desired result is that some of the fibers of at least one of the webs are forced into the adjacent web or layer thereby causing fiber entanglement between the interfaces of the two surfaces so as to result in the bonding or joining of the webs/layers together due to the increased entanglement of the fibers. The degree of bonding or entanglement will depend on a number of factors including, but not limited to, the types of fibers being used, the fiber lengths, the degree of pre-bonding or entanglement of the web or webs prior to subjection to the fluid entangling process, the type of fluid being used (liquids, such as water, steam or gases, such as air), the pressure of the fluid, the number of fluid streams, the speed of the process, the dwell time of the fluid and the porosity of the web or webs/other layers and the forming/carrier surface. One of the most common fluid entangling processes is referred to as hydroentangling which is a well-known process to those of ordinary skill in the art of nonwoven webs. Examples of fluid entangling process can be found in U.S. Pat. No. 4,939,016 to Radwanski et al., U.S. Pat. No. 3,485,706 to Evans, and U.S. Pat. Nos. 4,970,104 and 4,959,531 to Radwanski, each of which is incorporated herein in its entirety by reference thereto for all purposes.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

The term “user” refers herein to one who fits an absorbent article, such as, but not limited to, a diaper, diaper pant, training pant, youth pant, incontinent product, or other absorbent article about the wearer of one of these absorbent articles. A user and a wearer can be one and the same person.

### Absorbent Article:

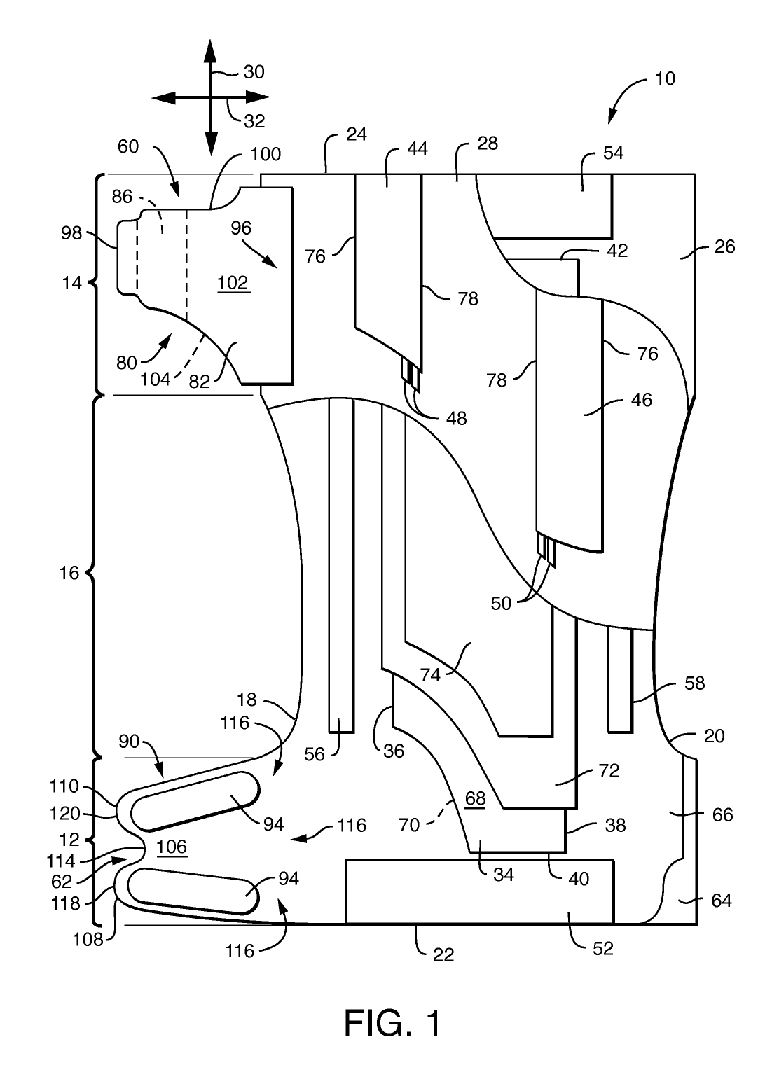

Referring to FIG. 1, a non-limiting illustration of an absorbent article 10, such as, for example, a diaper, is illustrated in a top down view with portions cut away for clarity of illustration. While the embodiments and illustrations described herein may generally apply to absorbent articles manufactured in the product longitudinal direction, which is hereinafter called the machine direction manufacturing of a product, it should be noted that one of ordinary skill could apply the information herein to absorbent articles manufactured in the latitudinal direction of the product which hereinafter is called the cross direction manufacturing of a product without departing from the spirit and scope of the disclosure. The absorbent article 10 illustrated in FIG. 1 includes a front waist region 12, a back waist region 14, and a crotch region 16 interconnecting the front and back waist regions, 12 and 14, respectively. The absorbent article 10 has a pair of longitudinal side edges, 18 and 20, and a pair of opposite waist edges, respectively designated front waist edge 22 and back waist edge 24. The front waist region 12 can be contiguous with the front waist edge 22 and the back waist region 14 can be contiguous with the back waist edge 24.

The absorbent article 10 can include an outer cover 26 and a body facing liner 28. In an embodiment, the body facing liner 28 can be bonded to the outer cover 26 in a superposed relation by any suitable means such as, but not limited to, adhesives, ultrasonic bonds, thermal bonds, pressure bonds, or other conventional techniques. The outer cover 26 can define a length, or longitudinal direction 30, and a width, or lateral direction 32, which, in the illustrated embodiment, can coincide with the length and width of the absorbent article 10.

An absorbent body 34 can be disposed between the outer cover 26 and the body facing liner 28. The absorbent body 34 can have longitudinal edges, 36 and 38, which, in an embodiment, can form portions of the longitudinal side edges, 18 and 20, respectively, of the absorbent article 10 and can have opposite end edges, 40 and 42, which, in an embodiment, can form portions of the waist edges, 22 and 24, respectively, of the absorbent article 10. In an embodiment, the absorbent body 34 can have a length and width that are the same as or less than the length and width of the absorbent article 10. In an embodiment, a pair of containment flaps, 44 and 46, can be present and can inhibit the lateral flow of body exudates.

The front waist region 12 can include the portion of the absorbent article 10 that, when worn, is positioned at least in part on the front of the wearer while the back waist region 14 can include the portion of the absorbent article 10 that, when worn, is positioned at least in part on the back of the wearer. The crotch region 16 of the absorbent article 10 can include the portion of the absorbent article 10, that, when worn, is positioned between the legs of the wearer and can partially cover the lower torso of the wearer. The waist edges, 22 and 24, of the absorbent article 10 are configured to enclose the waist of the wearer and together define the central waist opening. Portions of the longitudinal side edges, 18 and 20, in the crotch region 16 can generally define leg openings when the absorbent article 10 is worn.

The absorbent article 10 can be configured to contain and/or absorb liquid, solid, and semi-solid body exudates discharged from the wearer. For example, containment flaps, 44 and 46, can be configured to provide a barrier to the lateral flow of body exudates. A flap elastic member, 48 and 50, can be operatively joined to each containment flap, 44 and 46, in any suitable manner known in the art. The elasticized containment flaps, 44 and 46, can define a partially unattached edge that can assume an upright configuration in at least the crotch region 16 of the absorbent article 10 to form a seal against the wearer's body. The containment flaps, 44 and 46, can be located along the absorbent article 10 longitudinal side edges, 18 and 20, and can extend longitudinally along the entire length of absorbent article 10 or can extend partially along the length of the absorbent article 10. Suitable construction and arrangements for containment flaps, 44 and 46, are generally well known to those skilled in the art and are described in U.S. Pat. No. 4,704,116 issued Nov. 3, 1987, to Enloe and U.S. Pat. No. 5,562,650 issued Oct. 8, 1996 to Everett et al., which are incorporated herein by reference.

To further enhance containment and/or absorption of body exudates, the absorbent article 10 can suitably include a front waist elastic member 52, a back waist elastic member 54, and leg elastic members, 56 and 58, as are known to those skilled in the art. The waist elastic members, 52 and 54, can be attached to the outer cover 26 and/or the body facing liner 28 along the opposite waist edges, 22 and 24, and can extend over part or all of the waist edges, 22 and 24. The leg elastic members, 56 and 58, can be attached to the outer cover 26 and/or the body facing liner 28 along the opposite longitudinal side edges, 18 and 20, and positioned in the crotch region 16 of the absorbent article 10.

To maintain the absorbent article 10 in a closed configuration about the lower torso of a wearer, the absorbent article 10 can have at least two fastening systems, 60 and 62. The absorbent article 10 can include a back fastening system 60 and a front fastening system 62.

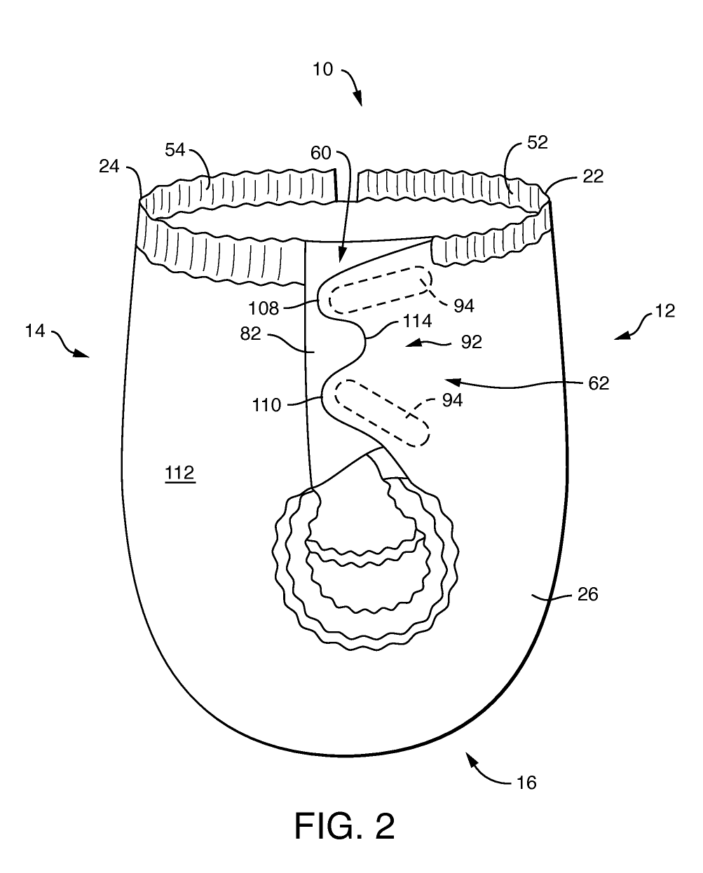

Additional details regarding each of these elements of the absorbent article 10 described herein can be found below and with reference to the FIGS. 1 and 2.

### Outer Cover:

The outer cover 26 can be breathable and/or liquid impermeable. The outer cover 26 can be elastic, stretchable or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the outer cover 26 can be constructed of a microporous polymeric film, such as polyethylene or polypropylene.

In an embodiment, the outer cover 26 can be a single layer of a liquid impermeable material. In an embodiment, the outer cover 26 can be suitably stretchable, and more suitably elastic, in at least the lateral or circumferential direction 32 of the absorbent article 10. In an embodiment, the outer cover 26 can be stretchable, and more suitably elastic, in both the lateral 32 and the longitudinal 30 directions. In an embodiment, the outer cover 26 can be a multi-layered laminate in which at least one of the layers is liquid impermeable. In an embodiment, the outer cover 26 may be a two layer construction, including an outer layer 64 material and an inner layer 66 material which can be bonded together such as by a laminate adhesive. Suitable laminate adhesives can be applied continuously or intermittently as beads, a spray, parallel swirls, or the like. Suitable adhesives can be obtained from Bostik Findlay Adhesives, Inc. of Wauwatosa, Wis., U.S.A. It is to be understood that the inner layer 66 can be bonded to the outer layer 64 utilizing ultrasonic bonds, thermal bonds, pressure bonds, or the like.

The outer layer 64 of the outer cover 26 can be any suitable material and may be one that provides a generally cloth-like texture or appearance to the wearer. An example of such material can be a 100% polypropylene bonded-carded web with a diamond bond pattern available from Sandler A.G., Germany, such as 30 gsm Sawabond 4185® or equivalent. Another example of material suitable for use as an outer layer 64 of an outer cover 26 can be a 20 gsm spunbond polypropylene non-woven web. The outer layer 64 may also be constructed of the same materials from which the body facing liner 28 can be constructed as described herein.

The liquid impermeable inner layer 66 of the outer cover 26 (or the liquid impermeable outer cover 26 where the outer cover 26 is of a single-layer construction) can be either vapor permeable (i.e., “breathable”) or vapor impermeable. The liquid impermeable inner layer 66 (or the liquid impermeable outer cover 26 where the outer cover 26 is of a single-layer construction) may be manufactured from a thin plastic film, although other liquid impermeable materials may also be used. The liquid impermeable inner layer 66 (or the liquid impermeable outer cover 26 where the outer cover 26 is of a single-layer construction) can inhibit liquid body exudates from leaking out of the absorbent article 10 and wetting articles, such as bed sheets and clothing, as well as the wearer and caregiver. An example of a material for a liquid impermeable inner layer 66 (or the liquid impermeable outer cover 26 where the outer cover 26 is of a single-layer construction) can be a printed 19 gsm Berry Plastics XP-8695H film or equivalent commercially available from Berry Plastics Corporation, Evansville, Ind., U.S.A.

Where the outer cover 26 is of a single layer construction, it can be embossed and/or matte finished to provide a more cloth-like texture or appearance. The outer cover 26 can permit vapors to escape from the absorbent article 10 while preventing liquids from passing through. A suitable liquid impermeable, vapor permeable material can be composed of a microporous polymer film or a non-woven material which has been coated or otherwise treated to impart a desired level of liquid impermeability.

### Absorbent Body:

The absorbent body 34 can be suitably constructed to be generally compressible, conformable, pliable, non-irritating to the wearer's skin and capable of absorbing and retaining liquid body exudates. The absorbent body 34 can be manufactured in a wide variety of sizes and shapes (for example, rectangular, trapezoidal, T-shape, I-shape, hourglass shape, etc.) and from a wide variety of materials. The size and the absorbent capacity of the absorbent body 34 should be compatible with the size of the intended wearer and the liquid loading imparted by the intended use of the absorbent article 10. Additionally, the size and the absorbent capacity of the absorbent body 34 can be varied to accommodate wearers ranging from infants to adults.

The absorbent body 34 may have a length ranging from about 150, 160, 170, 180, 190, 200, 210, 220, 225, 230, 240, 250, 260, 270, 280, 290, 300, 310, 320, 330, 340, or 350 mm to about 355, 360, 380, 385, 390, 395, 400, 410, 415, 420, 425, 440, 450, 460, 480, 500, 510, or 520 mm. The absorbent body 34 may have a crotch region 16 width ranging from about 30, 40, 50, 55, 60, 65, or 70 mm to about 75, 80, 85, 90, 95, 100, 105, 110, 115, 120, 125, 130, 140, 150, 160, 170 or 180 mm. The width of the absorbent body 34 located within the front waist region 12 and/or the back waist region 14 of the absorbent article 10 may range from about 50, 55, 60, 65, 70, 75, 80, 85, 90, or 95 mm to about 100, 105, 110, 115, 120, 125 or 130 mm. As noted herein, the absorbent body 34 can have a length and width that can be less than or equal to the length and width of the absorbent article 10.

In an embodiment, the absorbent article 10 can be a diaper having the following ranges of lengths and widths of an absorbent body 34 having an hourglass shape: the length of the absorbent body 34 may range from about 170, 180, 190, 200, 210, 220, 225, 240 or 250 mm to about 260, 280, 300, 310, 320, 330, 340, 350, 355, 360, 380, 385, or 390 mm; the width of the absorbent body 34 in the crotch region 16 may range from about 40, 50, 55, or 60 mm to about 65, 70, 75, or 80 mm; the width of the absorbent body 34 in the front waist region 12 and/or the back waist region 14 may range from about 80, 85, 90, or 95 mm to about 100, 105, or 110 mm.

In an embodiment, the absorbent article 10 can be an adult incontinence garment having the following ranges of lengths and widths of an absorbent body 34 having a rectangular shape: the length of the absorbent body 34 may range from about 400, 410 or 415 to about 425 or 450 mm; the width of the absorbent body 34 in the crotch region 16 may range from about 90, or 95 mm to about 100, 105, or 110 mm. It should be noted that the absorbent body 34 of an adult incontinence garment may or may not extend into either or both the front waist region 12 or the back waist region 14 of the absorbent article 10.

The absorbent body 34 can have two surfaces such as a wearer facing surface 68 and a garment facing surface 70. Edges, such as longitudinal side edges, 36 and 38, and such as front and back end edges, 40 and 42, can connect the two surfaces, 68 and 70.

In an embodiment, the absorbent body 34 can be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an embodiment, the absorbent body 34 can be a matrix of cellulosic fluff and superabsorbent material.

In an embodiment, the absorbent body 34 may be constructed of a single layer of materials, or in the alternative, may be constructed of two or more layers of materials. In an embodiment in which the absorbent body 34 has two layers, the absorbent body 34 can have a wearer facing layer suitably composed of hydrophilic fibers and a garment facing layer suitably composed at least in part of a high absorbency material commonly known as superabsorbent material. In such an embodiment, the wearer facing layer of the absorbent body 34 can be suitably composed of cellulosic fluff, such as wood pulp fluff, and the garment facing layer of the absorbent body 34 can be suitably composed of superabsorbent material, or a mixture of cellulosic fluff and superabsorbent material. As a result, the wearer facing layer can have a lower absorbent capacity per unit weight than the garment facing layer. The wearer facing layer may alternatively be composed of a mixture of hydrophilic fibers and superabsorbent material, as long as the concentration of superabsorbent material present in the wearer facing layer is lower than the concentration of superabsorbent material present in the garment facing layer so that the wearer facing layer can have a lower absorbent capacity per unit weight than the garment facing layer. It is also contemplated that, in an embodiment, the garment facing layer may be composed solely of superabsorbent material without departing from the scope of this disclosure. It is also contemplated that, in an embodiment, each of the layers, the wearer facing and garment facing layers, can have a superabsorbent material such that the absorbent capacities of the two superabsorbent materials can be different and can provide the absorbent body 34 with a lower absorbent capacity in the wearer facing layer than in the garment facing layer.

Various types of wettable, hydrophilic fibers can be used in the absorbent body 34. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. For example, one suitable type of fiber is a wood pulp that is a bleached, highly absorbent sulfate wood pulp containing primarily soft wood fibers. However, the wood pulp can be exchanged with other fiber materials, such as synthetic, polymeric, or meltblown fibers or with a combination of meltblown and natural fibers. In an embodiment, the cellulosic fluff can include a blend of wood pulp fluff. An example of wood pulp fluff can be “CoosAbsorb™ S Fluff Pulp” or equivalent available from Abitibi Bowater, Greenville, S.C., U.S.A., which is a bleached, highly absorbent sulfate wood pulp containing primarily southern soft wood fibers.

The absorbent body 34 can be formed with a dry-forming technique, an air-forming technique, a wet-forming technique, a foam-forming technique, or the like, as well as combinations thereof. A coform nonwoven material may also be employed. Methods and apparatus for carrying out such techniques are well known in the art.

Suitable superabsorbent materials can be selected from natural, synthetic, and modified natural polymers and materials. The superabsorbent materials can be inorganic materials, such as silica gels, or organic compounds, such as cross-linked polymers. Cross-linking may be covalent, ionic, Van der Waals, or hydrogen bonding. Typically, a superabsorbent material can be capable of absorbing at least about ten times its weight in liquid. In an embodiment, the superabsorbent material can absorb more than twenty-four times its weight in liquid. Examples of superabsorbent materials include polyacrylamides, polyvinyl alcohol, ethylene maleic anhydride copolymers, polyvinyl ethers, hydroxypropyl cellulose, carboxymal methyl cellulose, polyvinylmorpholinone, polymers and copolymers of vinyl sulfonic acid, polyacrylates, polyacrylamides, polyvinyl pyrrolidone, and the like. Additional polymers suitable for superabsorbent material include hydrolyzed, acrylonitrile grafted starch, acrylic acid grafted starch, polyacrylates and isobutylene maleic anhydride copolymers and mixtures thereof. The superabsorbent material may be in the form of discrete particles. The discrete particles can be of any desired shape, for example, spiral or semi-spiral, cubic, rod-like, polyhedral, etc. Shapes having a largest greatest dimension/smallest dimension ratio, such as needles, flakes, and fibers are also contemplated for use herein. Conglomerates of particles of superabsorbent materials may also be used in the absorbent body 34.

In an embodiment, the absorbent body 34 can be free of superabsorbent material. In an embodiment, the absorbent body 34 can have at least about 15% by weight of a superabsorbent material. In an embodiment, the absorbent body 34 can have at least about 15, 20, 25, 30, 35, 40, 45, 50, 55, 60, 65, 70, 75, 80, 85, 90, 95, 99 or 100% by weight of a superabsorbent material. In an embodiment, the absorbent body 34 can have less than about 100, 99, 95, 90, 85, 80, 75, 70, 65, 60, 55, 50, 45, 40, 35, 30, 25, or 20% by weight of a superabsorbent material. In an embodiment, the absorbent body 34 can have from about 15, 20, 25, 30, 35, 40, 45, 50, 55 or 60% to about 65, 70, 75, 80, 85, 90, 95, 99 or 100% by weight of a superabsorbent material. Examples of superabsorbent material include, but are not limited to, FAVOR SXM-9300 or equivalent available from Evonik Industries, Greensboro, N.C., U.S.A. and HYSORB 8760 or equivalent available from BASF Corporation, Charlotte, N.C., U.S.A.

The absorbent body 34 can be superposed over the inner layer 66 of the outer cover 26, extending laterally between the leg elastic members, 56 and 58, and can be bonded to the inner layer 66 of the outer cover 26, such as by being bonded thereto with adhesive. However, it is to be understood that the absorbent body 34 may be in contact with, and not bonded with, the outer cover 26 and remain within the scope of this disclosure. In an embodiment, the outer cover 26 can be composed of a single layer and the absorbent body 34 can be in contact with the singer layer of the outer cover 26. In an embodiment, a layer, such as but not limited to, a fluid transfer layer 72, can be positioned between the absorbent body 34 and the outer cover 26.

### Fluid Transfer Layer:

In various embodiments an absorbent article 10 can be constructed without a fluid transfer layer 72. In various embodiments the absorbent article 10 can have a fluid transfer layer 72. In an embodiment, the fluid transfer layer 72 can be in contact with the absorbent body 34. In an embodiment, the fluid transfer layer 72 can be bonded to the absorbent body 34. Bonding of the fluid transfer layer 72 to the absorbent body 34 can occur via any means known to one of ordinary skill, such as, but not limited to, adhesives. In an embodiment, a fluid transfer layer 72 can be positioned between the body facing liner 28 and the absorbent body 34. In an embodiment, a fluid transfer layer 72 can completely encompass the absorbent body 34 and can be sealed to itself. In such an embodiment, the fluid transfer layer 72 may be folded over on itself and then sealed using, for example, heat and/or pressure. In an embodiment a fluid transfer layer 72 may be composed of separate sheets of material which can be utilized to partially or fully encompass the absorbent body 34 and which can be sealed together using a sealing means such as an ultrasonic bonder or other thermochemical bonding means or the use of an adhesive.

In an embodiment, the fluid transfer layer 72 can be in contact with and/or bonded with the wearer facing surface 68 of the absorbent body 34. In an embodiment, the fluid transfer layer 72 can be in contact with and/or bonded with the wearer facing surface and at least one of the edges, 36, 38, 40, and/or 42, of the absorbent body 34. In an embodiment, the fluid transfer layer 72 can be in contact with and/or bonded with the wearer facing surface 68, at least one of the edges, 36, 38, 40, and/or 42, and the garment facing surface 70 of the absorbent body 34. In an embodiment, the absorbent body 34 may be partially or completely encompassed by a fluid transfer layer 72.

The fluid transfer layer 72 can be pliable, less hydrophilic than the absorbent body 34, and sufficiently porous to thereby permit liquid body exudates to penetrate through the fluid transfer layer 72 to reach the absorbent body 34. In an embodiment, the fluid transfer layer 72 can have sufficient structural integrity to withstand wetting thereof and of the absorbent body 34. In an embodiment, the fluid transfer layer 72 can be constructed from a single layer of material or it may be a laminate constructed from two or more layers of material. In an embodiment, the fluid transfer layer 72 can include, but is not limited to, natural and synthetic fibers such as, but not limited to, polyester, polypropylene, acetate, nylon, polymeric materials, cellulosic materials such as wood pulp, cotton, rayon, viscose, LYOCELL® such as from Lenzing Company of Austria, or mixtures of these or other cellulosic fibers, and combinations thereof. Natural fibers can include, but are not limited to, wool, cotton, flax, hemp, and wood pulp. Wood pulps can include, but are not limited to, standard softwood fluffing grade such as “CoosAbsorb™ S Fluff Pulp” or equivalent available from Abitibi Bowater, Greenville, S.C., U.S.A., which is a bleached, highly absorbent sulfate wood pulp containing primarily southern soft wood fibers.

In various embodiments, the fluid transfer layer 72 can include cellulosic material. In various embodiments, the fluid transfer layer 72 can be creped wadding or a high-strength tissue. In various embodiments, the fluid transfer layer 72 can include polymeric material. In an embodiment, a fluid transfer layer 72 can include a spunbond material. In an embodiment, a fluid transfer layer 72 can include a meltblown material. In an embodiment, the fluid transfer layer 72 can be a laminate of a meltblown nonwoven material having fine fibers laminated to at least one spunbond nonwoven material layer having coarse fibers. In such an embodiment, the fluid transfer layer 72 can be a spunbond-meltblown (“SM”) material. In an embodiment, the fluid transfer layer 72 can be a spunbond-meltblown-spunbond (“SMS”) material. A non-limiting example of such a fluid transfer layer 72 can be a 10 gsm spunbond-meltblown-spunbond material. In various embodiments, the fluid transfer layer 72 can be composed of at least one material which has been hydraulically entangled into a nonwoven substrate. In various embodiments, the fluid transfer layer 72 can be composed of at least two materials which have been hydraulically entangled into a nonwoven substrate. In various embodiments, the fluid transfer layer 72 can have at least three materials which have been hydraulically entangled into a nonwoven substrate. A non-limiting example of a fluid transfer layer 72 can be a 33 gsm hydraulically entangled substrate. In such an example, the fluid transfer layer 72 can be a 33 gsm hydraulically entangled substrate composed of a 12 gsm spunbond material, a 10 gsm wood pulp material having a length from about 0.6 cm to about 5.5 cm, and an 11 gsm polyester staple fiber material. To manufacture the fluid transfer layer 72 just described, the 12 gsm spunbond material can provide a base layer while the 10 gsm wood pulp material and the 11 gsm polyester staple fiber material can be homogeneously mixed together and deposited onto the spunbond material and then hydraulically entangled with the spunbond material.

In various embodiments, a wet strength agent can be included in the fluid transfer layer 72. A non-limiting example of a wet strength agent can be Kymene 6500 (557LK) or equivalent available from Ashland Inc. of Ashland, Ky., U.S.A. In various embodiments, a surfactant can be included in the fluid transfer layer 72. In various embodiments, the fluid transfer layer 72 can be hydrophilic. In various embodiments, the fluid transfer layer 72 can be hydrophobic and can be treated in any manner known in the art to be made hydrophilic.

In an embodiment, the fluid transfer layer 72 can be in contact with and/or bonded with an absorbent body 34 which is made at least partially of particulate material such as superabsorbent material. In an embodiment in which the fluid transfer layer 72 at least partially or completely encompasses the absorbent body 34, the fluid transfer layer 72 should not unduly expand or stretch as this might cause the particulate material to escape from the absorbent body 34. In an embodiment, the fluid transfer layer 72, while in a dry state, should have respective extension values at peak load in the machine and cross directions of 30 percent or less and 40 percent or less, respectively.

In an embodiment, the fluid transfer layer 72 may have a longitudinal length the same as, greater than, or less than the longitudinal length of the absorbent body 34. The fluid transfer layer 72 can have a longitudinal length ranging from about 150, 160, 170, 180, 190, 200, 210, 220, 225, 230, 240, 250, 260, 270, 280, 290, 300, 310, 320, 330, 340, or 350 mm to about 355, 360, 380, 385, 390, 395, 400, 410, 415, 420, 425, 440, 450, 460, 480, 500, 510, or 520 mm.

### Acquisition Layer:

In various embodiments, the absorbent article 10 can have an acquisition layer 74. The acquisition layer 74 can help decelerate and diffuse surges or gushes of liquid body exudates penetrating the body facing liner 28. In an embodiment, the acquisition layer 74 can be positioned between the body facing liner 28 and the absorbent body 34 to take in and distribute body exudates for absorption by the absorbent body 34. In an embodiment, the acquisition layer 74 can be positioned between the body facing liner 28 and a fluid transfer layer 72 if a fluid transfer layer 72 is present.

In an embodiment, the acquisition layer 74 can be in contact with and/or bonded with the body facing liner 28. In an embodiment in which the acquisition layer 74 is bonded with the body facing liner 28, bonding of the acquisition layer 74 to the body facing liner 28 can occur through the use of an adhesive and/or point fusion bonding. The point fusion bonding can be selected from, but is not limited to, ultrasonic bonding, pressure bonding, thermal bonding, and combinations thereof. In an embodiment, the point fusion bonding can be provided in any pattern as deemed suitable.

The acquisition layer 74 may have any longitudinal length dimension as deemed suitable. The acquisition layer 74 may have a longitudinal length from about 120, 130, 140, 150, 160, 170, 180, 190, 200, 210, 220, 225, 230, 240, or 250 mm to about 260, 270, 280, 290, 300, 310, 320, 340, 350, 360, 380, 400, 410, 415, 420, 425, 440, 450, 460, 480, 500, 510 or 520 mm. In an embodiment, the acquisition layer 74 can have any length such that the acquisition layer 74 can be coterminous with the waist edges, 22 and 24, of the absorbent article 10.

In an embodiment, the longitudinal length of the acquisition layer 74 can be the same as the longitudinal length of the absorbent body 34. In such an embodiment the midpoint of the longitudinal length of the acquisition layer 74 can substantially align with the midpoint of the longitudinal length of the absorbent body 34.

In an embodiment, the longitudinal length of the acquisition layer 74 can be shorter than the longitudinal length of the absorbent body 34. In such an embodiment, the acquisition layer 74 may be positioned at any desired location along the longitudinal length of the absorbent body 34. As an example of such an embodiment, the absorbent article 10 may contain a target area where repeated liquid surges typically occur in the absorbent article 10. The particular location of a target area can vary depending on the age and gender of the wearer of the absorbent article 10. For example, males tend to urinate further toward the front region of the absorbent article 10 and the target area may be phased forward within the absorbent article 10. For example, the target area for a male wearer may be positioned about 2¾″ forward of the longitudinal midpoint of the absorbent body 34 and may have a length of about ±3″ and a width of about ±2″. The female target area can be located closer to the center of the crotch region 16 of the absorbent article 10. For example, the target area for a female wearer may be positioned about 1″ forward of the longitudinal midpoint of the absorbent body 34 and may have a length of about ±3″ and a width of about ±2″. As a result, the relative longitudinal placement of the acquisition layer 74 within the absorbent article 10 can be selected to best correspond with the target area of either or both categories of wearers.

In an embodiment, the absorbent article 10 may contain a target area centered within the crotch region 16 of the absorbent article 10 with the premise that the absorbent article 10 would be worn by a female wearer. The acquisition layer 74, therefore, may be positioned along the longitudinal length of the absorbent article 10 such that the acquisition layer 74 can be substantially aligned with the target area of the absorbent article 10 intended for a female wearer. Alternatively, the absorbent article 10 may contain a target area positioned between the crotch region 16 and the front waist region 12 of the absorbent article 10 with the premise that the absorbent article 10 would be worn by a male wearer. The acquisition layer 74, therefore, may be positioned along the longitudinal length of the absorbent article 10 such that the acquisition layer 74 can be substantially aligned with the target area of the absorbent article 10 intended for a male wearer.

In an embodiment, the acquisition layer 74 can have a size dimension that is the same size dimension as the target area of the absorbent article 10 or a size dimension greater than the size dimension of the target area of the absorbent article 10. In an embodiment, the acquisition layer 74 can be in contact with and/or bonded with the body facing liner 28 at least partially in the target area of the absorbent article 10.

In various embodiments, the acquisition layer 74 can have a longitudinal length shorter than, the same as, or longer than the longitudinal length of the absorbent body 34. In an embodiment in which the absorbent article 10 is a diaper, the acquisition layer 74 may have a longitudinal length from about 120, 130, 140, 150, 160, 170, or 180 mm to about 200, 210, 220, 225, 240, 260, 280, 300, 310 or 320 mm. In such an embodiment, the acquisition layer 74 may be shorter in longitudinal length than the longitudinal length of the absorbent body 34 and may be phased from the front end edge 40 of the absorbent body 34 a distance of from about 15, 20, or 25 mm to about 30, 35 or 40 mm. In an embodiment in which the absorbent article 10 is an adult incontinence garment, the acquisition layer 74 may have a longitudinal length from about 200, 210, 220, 230, 240, or 250 mm to about 260, 270, 280, 290, 300, 320, 340, 360, 380, 400, 410, 415, 425, or 450 mm. In such an embodiment, the acquisition layer 74 may have a longitudinal length shorter than the longitudinal length of the absorbent body 34 and the acquisition layer 74 may be phased a distance of from about 20, 25, 30 or 35 mm to about 40, 45, 50, 55, 60, 65, 70 or 75 mm from the front end edge 40 of the absorbent body 34.

The acquisition layer 74 may have any width as desired. The acquisition layer 74 may have a width dimension from about 15, 20, 25, 30, 35, 40, 45, 50, 55, 60, or 70 mm to about 80, 90, 100, 110, 115, 120, 130, 140, 150, 160, 170, or 180 mm. The width of the acquisition layer 74 may vary dependent upon the size and shape of the absorbent article 10 within which the acquisition layer 74 will be placed. The acquisition layer 74 can have a width smaller than, the same as, or larger than the width of the absorbent body 34. Within the crotch region 16 of the absorbent article 10, the acquisition layer 74 can have a width smaller than, the same as, or larger than the width of the absorbent body 34.

In an embodiment, the acquisition layer 74 can include natural fibers, synthetic fibers, superabsorbent material, woven material, nonwoven material, wet-laid fibrous webs, a substantially unbounded airlaid fibrous web, an operatively bonded, stabilized-airlaid fibrous web, or the like, as well as combinations thereof. In an embodiment, the acquisition layer 74 can be formed from a material that is substantially hydrophobic, such as a nonwoven web composed of polypropylene, polyethylene, polyester, and the like, and combinations thereof.

### Body Facing Liner:

In various embodiments, the body facing liner 28 of the absorbent article 10 can overlay the absorbent body 34 and the outer cover 26 and can isolate the wearer's skin from liquid waste retained by the absorbent body 34. In various embodiments, a fluid transfer layer 72 can be positioned between the body facing liner 28 and the absorbent body 34. In various embodiments, an acquisition layer 74 can be positioned between the body facing liner 28 and the absorbent body 34 or a fluid transfer layer 72, if present. In various embodiments, the body facing liner 28 can be bonded to the acquisition layer 74, or the fluid transfer layer 72 if no acquisition layer 74 is present, via adhesive and/or by a point fusion bonding. The point fusion bonding may be selected from ultrasonic, thermal, pressure bonding, and combinations thereof.

In an embodiment, the body facing liner 28 can extend beyond the absorbent body 34 and/or a fluid transfer layer 72, and/or an acquisition layer 74 to overlay a portion of the outer cover 26 and can be bonded thereto by any method deemed suitable, such as, for example, by being bonded thereto by adhesive, to substantially enclose the absorbent body 34 between the outer cover 26 and the body facing liner 28. The body facing liner 28 may be narrower than the outer cover 26, but it is to be understood that the body facing liner 28 and the outer cover 26 may be of the same dimensions. It is also contemplated that the body facing liner 28 may not extend beyond the absorbent body 34 and/or may not be secured to the outer cover 26. The body facing liner 28 can be suitably compliant, soft feeling, and non-irritating to the wearer's skin and can be the same as or less hydrophilic than the absorbent body 34 to permit body exudates to readily penetrate through to the absorbent body 34 and provide a relatively dry surface to the wearer.

The body facing liner 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the body facing liner 28. The body facing liner 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof.

For example, the body facing liner 28 can be composed of a meltblown or spunbond web of polyolefin fibers. Alternatively, the body facing liner 28 can be a bonded-carded web composed of natural and/or synthetic fibers. The body facing liner 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire body facing liner 28 or it can be selectively applied to particular sections of the body facing liner 28. In an embodiment, a body facing liner 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a body facing liner 28 can be a spunbond substrate with a basis weight from about 10 or 12 to about 15 or 20 gsm. In an embodiment, a body facing liner 28 can be a 12 gsm spunbond-meltblown-spunbond substrate having 10% meltblown content applied between the two spunbond layers.

Although the outer cover 26 and body facing liner 28 can include elastomeric materials, it is contemplated that the outer cover 26 and the body facing liner 28 can be composed of materials which are generally non-elastomeric. In an embodiment, the body facing liner 28 can be stretchable, and more suitably elastic. In an embodiment, the body facing liner 28 can be suitably stretchable and more suitably elastic in at least the lateral or circumferential direction of the absorbent article 10. In other aspects, the body facing liner 28 can be stretchable, and more suitably elastic, in both the lateral and the longitudinal directions.

### Containment Flaps:

In an embodiment, containment flaps, 44 and 46, can be secured to the body facing liner 28 of the absorbent article 10 in a generally parallel, spaced relation with each other laterally inward of the longitudinal side edges, 18 and 20, to provide a barrier against the flow of body exudates to the leg openings. In an embodiment, the containment flaps, 44 and 46, can extend longitudinally from the front waist region 12 of the absorbent article 10, through the crotch region 16 to the back waist region 14 of the absorbent article 10. The containment flaps, 44 and 46, can be bonded to the body facing liner 28 by a seam of adhesive to define a fixed proximal end 76 of the containment flaps, 44 and 46.

The containment flaps, 44 and 46, can be constructed of a fibrous material which can be similar to the material forming the body facing liner 28. Other conventional material, such as polymer films, can also be employed. Each containment flap, 44 and 46, can have a moveable distal end 78 which can include flap elastics, such as flap elastics 48 and 50, respectively. Suitable elastic materials for the flap elastic, 48 and 50, can include sheets, strands or ribbons of natural rubber, synthetic rubber, or thermoplastic elastomeric materials.

The flap elastics, 48 and 50, as illustrated, can have two strands of elastomeric material extending longitudinally along the distal ends 78 of the containment flaps, 44 and 46, in generally parallel, spaced relation with each other. The elastic strands can be within the containment flaps, 44 and 46, while in an elastically contractible condition such that contraction of the strands gathers and shortens the distal ends 78 of the containment flaps, 44 and 46. As a result, the elastic strands can bias the distal ends 78 of each containment flap, 44 and 46, toward a position spaced from the proximal end 76 of the containment flaps, 44 and 46, so that the containment flaps, 44 and 46, can extend away from the body facing liner 28 in a generally upright orientation of the containment flaps, 44 and 46, especially in the crotch region 16 of the absorbent article 10, when the absorbent article 10 is fitted on the wearer. The distal end 78 of the containment flaps, 44 and 46, can be connected to the flap elastics, 48 and 50, by partially doubling the containment flap, 44 and 46, material back upon itself by an amount which can be sufficient to enclose the flap elastics, 48 and 50. It is to be understood, however, that the containment flaps, 44 and 46, can have any number of strands of elastomeric material and may also be omitted from the absorbent article 10 without departing from the scope of this disclosure.

### Leg Elastics:

Leg elastic members, 56 and 58, can be secured to the outer cover 26, such as by being bonded thereto by laminate adhesive, generally laterally inward of the longitudinal side edges, 18 and 20, of the absorbent article 10. In an embodiment, the leg elastic members, 56 and 58, may be disposed between the inner layer 66 and outer layer 64 of the outer cover 26 or between other layers of the absorbent article 10. A wide variety of elastic materials may be used for the leg elastic members, 56 and 58. Suitable elastic materials can include sheets, strands or ribbons of natural rubber, synthetic rubber, or thermoplastic elastomeric materials. The elastic materials can be stretched and secured to a substrate, secured to a gathered substrate, or secured to a substrate and then elasticized or shrunk, for example, with the application of heat, such that the elastic retractive forces are imparted to the substrate.

### Waist Elastic Members:

In an embodiment, the absorbent article 10 can have waist elastic members, 52 and 54, which can be formed of any suitable elastic material. In such an embodiment, suitable elastic materials can include, but are not limited to, sheets, strands or ribbons of natural rubber, synthetic rubber, or thermoplastic elastomeric polymers. The elastic materials can be stretched and bonded to a substrate, bonded to a gathered substrate, or bonded to a substrate and then elasticized or shrunk, for example, with the application of heat, such that elastic retractive forces are imparted to the substrate. It is to be understood, however, that the waist elastic members, 52 and 54, may be omitted from the absorbent article 10 without departing from the scope of this disclosure.

### Fastening System:

To maintain the absorbent article 10 in a closed configuration about the lower torso of a wearer, the absorbent article 10 can have at least two fastening systems, 60 and 62, such as back fastening system 60 and front fastening system 62. While configurations, elements and/or materials can be described herein as relating particularly to one of the back fastening system 60 or front fastening system 62, it is to be understood that such configurations, elements and/or materials can be present in either or both the back fastening system 60 and/or front fastening system 62. While the back fastening system 60 can be described herein as attaching to a body facing surface of the absorbent article 10 in the front waist region 12 of the absorbent article 10 and the front fastening system 62 can be described herein as attaching to a garment facing surface of the absorbent article 10 in the back waist region 14 of the absorbent article, it is to be understood that such attachments can be reversed. In an embodiment, therefore, the back fastening system 60 can attach to a garment facing surface of the absorbent article 10 in the front waist region 12 of the absorbent article 10 and the front fastening system 62 can attach to a body facing surface of the absorbent article 10 in the back waist region 14.

The back fastening system 60 can have a pair of structural members 80 which can enclose the waist of the wearer of the absorbent article 10 and can be releasably attached to the interior of the absorbent article 10 in the front waist region 12 when the absorbent article 10 is in a closed configuration. In other words, in an embodiment, each structural member 80 can be releasably attached to a body facing surface of the front waist region 12 of the absorbent article 10. In an embodiment, each structural member 80 can be releasably attached to the body facing liner 28 of the absorbent article 10.

Each structural member 80 can extend outward, in a transverse direction 32, beyond each longitudinal side edge, 18 and 20, in the back waist region 14 of the absorbent article 10. Each structural member 80 can have a proximal region 96 located adjacent one of the longitudinal edges, 18 and 20, of the absorbent article 10, a distal edge 98 spaced transversely outward from the proximal region 96, and an end edge 100 between the proximal region 96 and the distal edge 98. In an embodiment such as illustrated in FIG. 1, the end edge 100 does not have to form a portion of the back waist edge 24 of the absorbent article 10. In an embodiment, the end edge 100 of the structural member 80 can form a portion of the back waist edge 24 of the absorbent article 10. The structural member 80 can have a body facing surface 102 and a garment facing surface 104.

In an embodiment, the structural members 80 can be at least partially extensible in the lateral direction 32. In an embodiment, the structural members 80 can be at least partially elastically extensible in the lateral direction 32. The structural members 80 can have any size, shape, and configuration as deemed suitable. The length, width, and thickness of the structural members 80 can vary dependent upon the size of the intended wearer of the absorbent article 10. In an embodiment, the structural members 80 can have a size in the lateral direction 32 as deemed suitable to enable the structural members 80 to enclose the waist of the wearer of the absorbent article 10 and to releasably attach to a body facing surface of the front waist region 12 of the absorbent article 10.

In an embodiment, the structural members 80 can be integral with the back waist region 14 of the absorbent article 10 and can comprise a portion of the material constructing the outer cover 26, the body facing liner 28, and combinations thereof. In an embodiment, the structural members 80 can be a separate material or a combination of separate materials which can be bonded to the absorbent article 10. Non-limiting examples of materials which can be utilized to construct the structural members 80 can include woven materials, nonwoven materials, films including formed films and polymeric films, laminates such as, but not limited to, nonwoven laminates, film laminates, and combinations thereof. The structural members 80 can be bonded to the absorbent article 10 in any manner deemed suitable such as, but not limited to, continuous or intermittent bonding utilizing adhesive, heat bonding, pressure bonding, ultrasonic bonding, mechanical bonding, and combinations thereof. FIG. 1 provides a non-limiting illustration of an absorbent article 10 in which the structural members 80 can be a separate material which can be bonded to the absorbent article 10.

The front fastening system 62 can have a pair of ear portions 90 which can extend outward, in a transverse direction 32, beyond each longitudinal side edge, 18 and 20, in the front waist region 12 of the absorbent article 10. In an embodiment, each ear portion 90 can releasably attach to a garment facing surface of the absorbent article 10. In an embodiment, each of the ear portions 90 can releasably attach to the outer cover 26 in the back waist region 14 of the absorbent article 10 when the absorbent article 10 is secured about the lower torso of a wearer. In an embodiment, each of the ear portions 90 can releasably attach to a portion of the garment facing surface 104 of each of the structural members 80 of the back fastening system 60 when the absorbent article 10 is secured about the lower torso of a wearer.

In an embodiment, each ear portion 90 can be integral with the front waist region 12 of the absorbent article 10 and can comprise a portion of the material constructing the outer cover 26, the body facing liner 28, and combinations thereof. In an embodiment, the ear portions 90 can be a separate material or a combination of separate materials which can be bonded to the absorbent article 10. Non-limiting examples of materials which can be utilized to construct the ear portions 90 can include woven materials, nonwoven materials, films including formed films and polymeric films, laminates such as, but not limited to, nonwoven laminates, film laminates, and combinations thereof. In an embodiment, the ear portions 90 can be constructed of non-elastic material. In an embodiment, the ear portions 90 can be at least partially extensible in the lateral direction 32. In an embodiment, the ear portions 90 can be at least partially elastically extensible in the lateral direction 32. The ear portions 90 can be bonded to the absorbent article 10 in any manner deemed suitable such as, but not limited to, continuous or intermittent bonding utilizing adhesive, heat bonding, pressure bonding, ultrasonic bonding, mechanical bonding, and combinations thereof. FIG. 1 provides a non-limiting illustration of an absorbent article 10 in which the ear portions 90 can be integral with the outer cover 26 of the absorbent article 10 in the front waist region 12 of the absorbent article 10.

To provide for releasable attachment of each of the back fastening system 60 and the front fastening system 62, each of the back fastening system 60 and the front fastening system 62 can have fasteners. The fasteners can operate to maintain the absorbent article 10 in a closed configuration about the waist and legs of the wearer of the absorbent article 10. In an embodiment, the fasteners of each of the back fastening system 60 and front fastening system 62 can provide an engaging component which can releasably attach and engage with a landing component. A landing component can be a material or combination of materials which can engage with the fastener to provide a fastening assembly and which can maintain the absorbent article 10 in a closed configuration. A landing component can be integral with the absorbent article 10 or can be a material or combination of materials which can be bonded to the absorbent article 10 for the purpose of providing a landing component for a fastener. The fasteners of the back fastening system 60 and of the front fastening system 62 can include any attachment mechanism deemed suitable such as, but not limited to, pressure sensitive adhesives, cohesive materials, mechanical fasteners such as hooks, and any other type of fastener such as, but not limited to, magnets, buttons, buckles, snaps, or the like. The landing component can be any material or combination of materials which can cooperate with the fasteners to provide a fastening assembly. The landing component can include, but is not limited to, pressure sensitive adhesives, cohesive materials, mechanical fasteners, loop materials, magnets, button holes, snaps, or the like. In an embodiment, a landing component can be a portion of the material or combination of materials utilized to construct the outer cover 26, the body facing liner 28, a structural member 80 or any other material or combination of materials which can be bonded to the absorbent article 10 for the purpose of being a landing component.

In an embodiment, the fasteners of the back fastening system 60 and the front fastening assembly 62 can comprise hook fasteners. In such an embodiment, the hook fastener can include a base material and a plurality of engaging elements extending from the base material. The engaging elements of the hook fastener can engage fibrous elements of a landing component so as to provide a secure fastening assembly. The hook fastener can be manufactured from any material as deemed suitable such as, but not limited to, nylon, polyester, polyethylene, polypropylene, and combinations thereof. In an embodiment, the hook fastener can have shaped engaging elements projecting from a base material such as provided by “Velcro USA—Code HTH-877 (85-1126) 2×1 “Hook”” available from Velcro USA. The landing component can be a loop material which can include material as deemed suitable to engage with the engaging elements of the hook fastener. In an embodiment, the loop material can include a plurality of fibrous elements which can engage the engaging elements of the hook fastener. The loop material can be manufactured from any material deemed suitable to provide fibrous elements such as, but not limited to, nylon, polyester, polypropylene, an elastic laminate, and combinations thereof. It is to be understood that the loop material need not be a separate material or combinations of materials bonded to the absorbent article 10 for the purpose of providing a landing component. It is to be understood that the loop material can be a portion of any material which can provide a landing component for the hook fastener such as, but not limited to, any material or combination of materials utilized to construct the outer cover 26, the body facing liner 28, and a structural member 80.

In an embodiment, at least a portion of the fasteners of each of the back fastening system 60 and the front fastening system 62 can be located on either a garment facing surface or a body facing surface of the structural members 80 or ear portions 90. In an embodiment, at least a portion of the fasteners of each of the back fastening system 60 and the front fastening system 62 can be a discrete separate material bonded to the structural members 80 or ear portions 90, respectively, or can be integral with a material or combination of materials utilized to construct the structural members 80 or ear portions 90.

Referring to FIG. 1, at least a portion of the fasteners of the back fastening system 60 can be located on the garment facing surface 104 of the structural members 80. In an embodiment, at least a portion of the structural member 80 can have a stretch component 82. In an embodiment, the fasteners can be hook fasteners 86 which can include a hook base material component and engaging elements projecting from the base material component. The hook fastener 86 can be located between the distal edge 98 and the proximal region 96 of the structural member 80. The hook fasteners 86 can be any size and shape as deemed suitable to releasably attach to a body facing surface of the absorbent article 10. The hook fastener 86 can have a shape such as, but not limited to, rectangle, square, circle, oval, oblong, triangle, diamond, and any other shape deemed suitable. In the non-limiting illustration of FIG. 1, the hook fastener 86 can be illustrated in a generally rectangular shape, in the longitudinal direction 30, of the structural member 80. In an embodiment, the structural members 80 can have more than one hook fastener 86. In an embodiment, the structural members 80 can have at least 1, 2, 3, 4, 5 or 6 hook fasteners 86. As the hook fastener 86 can be located on the garment facing surface 104 of the structural members 80, when positioning the absorbent article 10 around the waist of the wearer of the absorbent article 10, the hook fastener 86 can engage a body facing surface of the absorbent article 10. In an embodiment, when positioning the absorbent article 10 around the waist of the wearer of the absorbent article 10, the hook fastener 86 of the structural member 80 can engage the body facing liner 28 in the front waist region 12 of the absorbent article 10. In an embodiment, when positioning the absorbent article 10 around the waist of the wearer of the absorbent article 10, the hook fastener 86 of the structural member 80 can engage a landing component (not shown) which can be a separate material or combination of materials bonded to the body facing liner 28 of the absorbent article 10 in the front waist region 12 of the absorbent article 10.

Referring to FIG. 1, at least a portion of the fasteners of the front fastening system 62 can be located on the body facing surface 106 of the ear portions 90. In an embodiment, at least a portion of the ear portions 90 can have a fastener located on the body facing surface 106 of the ear portions 90 laterally outward of the longitudinal edges, 18 and 20, of the absorbent article 10. In an embodiment, the fastener can be a hook fastener 94 which can include a hook base material component and engaging elements projecting from the base material component. The hook fastener 94 can be any size and shape as deemed suitable to releasably attach to a garment facing surface of the absorbent article 10. The hook fastener 94 can have a shape such as, but not limited to, rectangle, square, circle, oval, oblong, triangle, diamond, and any other shape deemed suitable. As illustrated in FIG. 1, the hook fastener 94 can be illustrated in a generally oblong shape. In an embodiment, the ear portions 90 can have more than one hook fastener 94. In an embodiment, the ear portions 90 can have at least 1, 2, 3, 4, 5 or 6 hook fasteners 94. As illustrated in FIG. 1, the ear portion 90 can have at least two hook fasteners 94. As the hook fasteners 94 can be located on the body facing surface 106 of the ear portions 90, when positioning the absorbent article 10 around the waist of the wearer of the absorbent article 10, the hook fasteners 94 can engage a garment facing surface of the absorbent article 10. In an embodiment, when positioning the absorbent article 10 around the waist of the wearer of the absorbent article 10, the hook fasteners 94 of the ear portions 90 can engage the garment facing surface 112 of the outer cover 26 in the back waist region 14 of the absorbent article 10. In an embodiment, when positioning the absorbent article 10 around the waist of the wearer of the absorbent article 10, the hook fasteners 94 of the ear portions 90 can engage the garment facing surface 104 of the structural members 80 of the back fastening system 60 of the absorbent article 10.

In an embodiment, the ear portion 90 of the front fastening system 62 can be shaped. In an embodiment, such as illustrated in the non-limiting illustration of FIG. 1, the ear portions 90 can be bilobate. In an embodiment, the ear portions 90 can have at least 2 (“bilobate”), 3 (“trilobate”), or 4 (“quadrilobate”) lobes. In an embodiment in which the ear portion 90 is bilobate, the ear portions 90 can have two lobes, 108 and 110, which can be separated from each other by a recessed area 114. Each lobe, 108 and 110, can be configured from and extend from material in common with each lobe, 108 and 110. Each lobe, 108 and 110, can, therefore, extend from a common base region 116. Each lobe, 108 and 110, can have a distal edge, 118 and 120, respectively. As illustrated in FIG. 1, the distal edges, 118 and 120, of the lobes, 108 and 110, are illustrated in a rounded configuration. It will be understood that the distal edges, 118 and 120, can have alternate configurations such as squared, angular, pointed, and the like.

In an embodiment, each of the lobes, 108 and 110, can have the same length in the transverse direction 32 of the absorbent article 10 as measured from the base region 116 to the distal edge, 118 or 120, of the lobe, 108 or 110, respectively. In an embodiment, one of the lobes, 108 or 110, can be longer than or shorter than the other lobe, 108 or 110, in the transverse direction 32 of the absorbent article 10 as measured from the base region 116 to the distal edge, 118 or 120, of the lobe, 108 or 110, respectively. The recessed area 114 can extend laterally inward toward the longitudinal side edges, 18 and 20, of the absorbent article 10 a distance up to about 5, 10, 15, 20, 25, 30, 35, 40, 45, 50, 55, 60, 65, 70 or 75% of the length of a lobe, 108 or 110, of the ear portion 90. In an embodiment, the recessed area 114 does not completely separate the material constructing the ear portion 90 into two segments of material. In other words, each lobe, 108 and 110, of each ear portion 90 is configured from and extends from a common base region 116.

In an embodiment, such as illustrated in FIG. 1, the ear portions 90 can have more than one hook fastener 94 and at least a portion of a hook fastener 94 can be located in each of the lobes, 108 and 110. In an embodiment, at least a portion of a hook fastener 94 can be located proximate the distal edges, 118 and 120, of the lobes, 108 and 110, respectively. In an embodiment, at least a portion of the hook fastener 94 of each lobe, 108 and 110, can have a size such that the hook fasteners 94 can extend into the common base region 116 without overlapping any other hook fastener 94 from any other lobe.

Without being bound by theory, it is believed that providing an ear portion 90 with multiple lobes, and each lobe having a hook fastener 94, can increase the ability of a user to provide a good fit of the absorbent article 10 on the wearer of the absorbent article 10. A bilobate ear portion 90 with at least a portion of a hook fastener 94 located in each lobe, 108 and 110, can provide easier maneuverability of the ear portion 90 to the user. For example, when the ear portion 90 has been attached to the back waist region 14 of the absorbent article 10, the user can releasably attach and re-attach a lobe, such as 108 or 110, of the ear portion 90 without having to disengage the entire ear portion 90 from the back waist region 14 of the absorbent article 10. In an embodiment, the recessed area 114 located between the lobes, 108 and 110, can provide a location for a user to insert their finger in order to attach and/or re-attach one or both of the lobes, 108 and/or 110. Repositioning of lobe 108 can provide an adjustment of the fit of the absorbent article 10 about the waist of the wearer of the absorbent article 10. Repositioning of lobe 110 can provide an adjustment of the fit of the absorbent article 10 about the leg of the wearer of the absorbent article 10. Without being bound by theory, it is believed that adjustment of lobe 110 can impact the positioning and/or the tensioning of a leg elastic, such as leg elastic 56, of the absorbent article 10.

Each lobe, such as lobes 108 and 110, can be adjusted and/or readjusted independently of the other lobe, such as lobe 108 and 110. In an embodiment, a user can adjust lobes 108 and 110 to adjust the fit of both the waist and the leg opening of the absorbent article. In an embodiment, a user can adjust only lobe 108 to adjust only the waist of the absorbent article 10. In an embodiment, a user can adjust only lobe 110 to adjust only the leg opening of the absorbent article 10. As described herein, the ear portion 90 can be releasably attached to a garment facing surface of the absorbent article 10. As a user is adjusting either of the lobes 108 and 110 of an ear portion 90, it should be understood that such adjustment could result in the lobe, 108 or 110, being reattached to a garment facing surface of the absorbent article 10 or to a garment facing surface of the ear portion 90 itself.

To apply the absorbent article 10 described herein to a wearer, a user can position the back waist region 14 of the absorbent article 10 behind the hips of the wearer with the front waist region 12 extended in a direction towards the wearer's legs. The front waist region 12 of the absorbent article 10 can be pulled between the wearer's legs and up to the wearer's waist. Each of the structural members 80 of the back fastening system 60 can be attached to a body facing surface of the absorbent article in the front waist region 12 of the absorbent article 10 such that the hook fastener 86 of each of the structural members 80 can be engaged with the body facing surface of the front waist region 12 of the absorbent article 10. Once in this position, the ear portions 90 of the front fastening system 62 can each engage a portion of a garment facing surface of the absorbent article 10. In an embodiment, the hook fasteners 94 of the ear portions 90 can engage a portion of the outer cover 26 of the absorbent article 10. In an embodiment, the hook fasteners 94 of the ear portions 90 can engage a portion of the garment facing surface 104 of the structural members 80. In such an embodiment, the hook fasteners 94 of the ear portions 90 can engage a portion of the stretch component 82 of the structural members 80.

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a. a front waist region, a back waist region, and a crotch region interconnecting the front waist region and the back waist region;
b. a garment facing surface and a body facing surface;
c. a back fastening system located in the back waist region comprising:
i. a pair of structural members having a garment facing surface and a body facing surface;
ii. a fastener located on the garment facing surface of each of the structural members and configured to releasably engage the body facing surface in the front waist region of the absorbent article;
d. a front fastening system located in the front waist region comprising:
i. a pair of bilobate ear portions having a garment facing surface and a body facing surface; and
ii. a fastener located on the body facing surface of each of the ear portions.

2. The absorbent article of claim 1 wherein the pair of ear portions of the front fastening system is constructed from a non-elastic material.

3. The absorbent article of claim 1 wherein the pair of ear portions of the front fastening system is constructed from an elastic material.

4. The absorbent article of claim 1 wherein the fastener of each of the structural members comprises a hook fastener.

5. The absorbent article of claim 1 wherein the fastener of each of the ear portions comprises a hook fastener.

6. The absorbent article of claim 1 wherein each lobe of each ear portion comprises a fastener.

7. The absorbent article of claim 1 wherein each of the structural members of the back fastening system further comprise a stretch component.

8. The absorbent article of claim 1 wherein the fastener of each of the ear portions is configured to releasably engage the garment facing surface of the absorbent article in the back waist region of the absorbent article.

9. The absorbent article of claim 1 wherein the fastener on each of the ear portions is configured to releasably engage the garment facing surface of each of the structural members of the back fastening system.

10. An absorbent article comprising:
a. a front waist region, a back waist region, and a crotch region interconnecting the front waist region and the back waist region;
b. a garment facing surface and a body facing surface;
c. an outer cover and a body facing liner;
d. a back fastening system located in the back waist region comprising:
i. a pair of structural members having a garment facing surface and a body facing surface;
ii. a fastener located on the garment facing surface of each of the structural members and configured to releasably engage the body facing surface in the front waist region of the absorbent article;
e. a front fastening system located in the front waist region comprising:
i. a pair of bilobate ear portions having a garment facing surface and a body facing surface; and
ii. a fastener at least partially located in each lobe of each ear portion and configured to releasably engage the outer cover.

11. The absorbent article of claim 10 wherein the pair of ear portions of the front fastening system is constructed from a non-elastic material.

12. The absorbent article of claim 10 wherein the pair of ear portions of the front fastening system is constructed from an elastic material.

13. The absorbent article of claim 10 wherein each of the structural members of the back fastening system further comprise a stretch component.

14. The absorbent article of claim 10 wherein the fastener of each of the structural members comprises a hook fastener.

15. The absorbent article of claim 10 wherein each of the fasteners of each of the lobes of each of the ear portions comprises a hook fastener.

16. An absorbent article comprising:
a. a front waist region, a back waist region, and a crotch region interconnecting the front waist region and the back waist region;
b. a garment facing surface and a body facing surface;
c. an outer cover and a body facing liner;
d. a back fastening system located in the back waist region comprising:
i. a pair of structural members having a garment facing surface and a body facing surface;
ii. a fastener located on the garment facing surface of each of the structural members and configured to releasably engage the body facing surface in the front waist region of the absorbent article;
e. a front fastening system located in the front waist region comprising:
i. a pair of bilobate ear portions having a garment facing surface and a body facing surface;
ii. a fastener at least partially located in each lobe of each ear portion and configured to releasably engage with the garment facing surface of each structural member.

17. The absorbent article of claim 16 wherein the pair of ear portions of the front fastening system is constructed from a non-elastic material.

18. The absorbent article of claim 16 wherein the pair of ear portions of the front fastening system is constructed from an elastic material.

19. The absorbent article of claim 16 wherein each of the structural members of the back fastening system further comprise a stretch component.

20. The absorbent article of claim 16 wherein the fastener of each of the structural members comprises a hook fastener.

21. The absorbent article of claim 16 wherein each of the fasteners of each of the lobes of each of the ear portions comprises a hook fastener.
